(19) United States
(12) Patent Application Publication
(10) Pub. No.: US 2015/0250404 A1
(43) Pub. Date: Sep. 10, 2015

(54) METHOD AND APPARATUS FOR DETECTION OF INSULIN RESISTANCE, DIABETES AND CARDIOVASCULAR DISEASE

(71) Applicant: MEDICAL SCREENING CORPORATION, Miami, FL (US)

(72) Inventor: Maarek Albert, Miami, FL (US)

(73) Assignees: MEDICAL SCREENING CORPORATION, Miami, FL (US); MEDICAL SCREENING COMPANY

(21) Appl. No.: 14/717,276

(22) Filed: May 20, 2015

Related U.S. Application Data
(63) Continuation-in-part of application No. PCT/IB2013/002595, filed on Nov. 21, 2013.
(60) Provisional application No. 61/728,848, filed on Nov. 21, 2012.

(51) Int. Cl. A61B 5/053; A61B 5/00; A61B 5/1455

(52) U.S. Cl. CPC A61B 5/0533; A61B 5/14552; A61B 5/7257

(57) ABSTRACT

A method to detect insulin resistance, diabetes and/or cardiovascular or autonomic neuropathy complications, the method comprising performing a spectral analysis using Fast Fourier Transformation (FFT) of the first derivative of total records of an oximeter wave form (plethysmograph), to obtain 3 frequencies; high (PTGHF), low (PTGLF) and very low (PTGVLF) frequencies wherein the sum of the 3 frequencies is the total power of the spectral analysis in ms² (PTG TP) and wherein the sum of the amplitude of the 3 frequencies is the PTG Index of the Spectral Analysis (PTGi).

The present invention relates to a method and apparatus to detect insulin resistance, diabetes and complications of diabetes such as cardiovascular diseases and autonomic neuropathy. More specifically the invention relates to the spectral analysis of arterial photo plethysmography to detect insulin resistance, diabetes and complications of diabetes such as cardiovascular diseases and autonomic neuropathy. Most specifically the invention relates to the use of an oximeter to detect insulin resistance, diabetes and complications of diabetes such as cardiovascular diseases and autonomic neuropathy.

The prevalence of type 2 diabetes mellitus (T2DM) has increased in recent decades to epidemic proportions. About 150 million individuals worldwide had T2DM in 2000, and this number is expected to increase to −300 million by the year 2025’. Because of the chronic course of T2DM and the significant morbidity and mortality associated with the vascular complications of the disease, T2DM has become not only a serious public health threat, but also a heavy economic burden on the health care system². The total annual cost of diabetes care in the United States was estimated to be $175 billion in the year 2007, and this number is expected to increase further with the increasing incidence of the disease.

The association of obesity with T2DM has been recognized for decades, and the major basis for this link is the ability of obesity to engender insulin resistance. Insulin resistance is a fundamental aspect of the etiology of T2DM and is also linked to a wide array of other pathophysiologic sequelae including hypertension, hyperlipidemia, atherosclerosis (i.e., the metabolic syndrome, or syndrome X), and polycystic ovarian disease^{3, 4}.

Insulin resistance carried a greater risk for developing cardiovascular disease than smoking or age or total/HDL cholesterol ratio^{5, 6, 7, 8}.

There are also grounds for considering the related possibility that insulin resistance and hyperinsulinemia, in addition to being caused by obesity, can contribute to the development of obesity⁹.

Type 2 diabetes can progress undetected for many years, causing cardiovascular diseases^{10 u}. By the time patients are diagnosed with diabetes, up to 50% of them have cardiovascular complications.

Recent studies indicate that early detection of diabetes cardiovascular complications can decrease diabetic mortality^{13, 14}. However, the early detection of cardiovascular diseases is made difficult because symptoms are very often absent in patients.¹⁴

Thus, the detection of insulin resistance, diabetes and cardiovascular complications could be useful in diabetes treatment management and early detection of its complications.

The diagnosis of insulin resistance requires performing of the gold standard hyperinsulinemic euglycemic clamp (HE Clamp) which is costly, time consuming and inconvenient in routine clinical setting¹⁵.

The diagnosis of diabetes uses the blood tests such as Fasting Plasma Glucose (FPG) and Oral Glucose Tolerance Tests (OGTT). Studies demonstrate that FPG has a very low sensitivity to detect Diabetes and OGTT is costly and time consuming (exam duration is from 2 to 5 hours).

The diagnosis of cardiovascular diseases uses EKG, Stress Testing, Echocardiography, Chest X ray, EBCT and other Coronary Angiography. There is no gold standard and all this battery of tests is costly and time consuming.

The diagnosis of autonomic neuropathy uses a battery of tests including Ewing tests, heart rate variability analysis, sudomotor function, Nerve conductance study, thermal stimulation and other skin biopsy. There is no gold standard and all this battery of tests is costly and time consuming.

It is an aim of the present invention to improve the ability to detect insulin resistance, diabetes detection and/or cardiovascular or autonomic neuropathy complication of diabetes that compares favorably with the standardized methods but is not expensive or time consuming to perform.

According to the present invention there is provided a method to detect insulin resistance, diabetes and/or cardiovascular or autonomic neuropathy complications, the method comprising analysis of the fast Fourier transformation (FFT) of the oximeter wave form (plethysmograph) using as reference the heart rate with frequency values fixed at 1 Hertz (Hz) at heart rate 60 beat per minute (bpm).

The spectral analysis using the Fast Fourier Transformation (FFT) of the first derivative of total records of the plethysmograph provides 3 frequencies; high (PTGHF), low (PTGLF) and very low frequencies (PTGVLF).

The sum of the surface of the 3 frequencies is the total Power of the spectral analysis (PTG TP).

The sum of the amplitude of the 3 frequencies is the PTG Index of the Spectral Analysis (PTGi).

The Ratio PTGVLF/PTG) is the PTG ratio (PTG r).

The algorithm using PTGVLF and a marker of the sudomotor function named ESRNO assessed by the SudoPath system (galvanic skin response) is the PTG VLFi.

According to the present invention there is provided an assay for use in the early detection of insulin resistance, diabetes and/or cardiovascular diseases or autonomic neuropathy, the assay comprising the steps of obtaining an oximeter plethysmograph trace for a patient, performing a fast Fourier transformation of the first derivative of total records of the plethysmograph trace and calculating the PTGTP in ms² (milliseconds squared), PTG HF, PTGVLF and PTG LF wherein a measurement of PTGTP greater than 370 ms² suggests the patient has insulin resistance.

PTGi lower than 40 suggests the patient has diabetes with high risk of cardiovascular disease.

PTGVLF i greater than 33 suggests the patient has diabetes with a high risk of autonomic neuropathy.

The invention also provides hardware comprising an oximeter and software installed on a PC to carry out the assay and analyze the results.

In one particular embodiment the invention further provides the use of the TM-Oxi and SudoPath systems in the detection of insulin resistance, diabetes and/or cardiovascular or autonomic neuropathy complications.

In addition to the oximeter, the TM-Oxi system further comprises a blood pressure device powered by the USB port of the PC.

The invention further provides the use of an oximeter in the detection of insulin resistance, diabetes and cardiovascular or autonomic neuropathy complications.

The invention thus provides a low cost, quickly performed test that gives results that correlate very highly with the HE Clamp gold standard test and standardized methods for detecting diabetes and risk of cardiovascular diseases and/or autonomic neuropathy.

The present invention is supported by 3 clinical trials as described in the specific examples below:

The correlation of M-value and PTG Total Power (PTGTP) using the Spearman's coefficient was −0.624 (P.0.001).

PTGTP had a sensitivity and specificity of 90% (cutoff #370 ms2) to detect M-value <4.5 (P.0.0001).

PTGi had a sensitivity of 86.1% and specificity of 87.3% (cutoff #<40.8) to detect atherosclerosis (P.0.0001). Area under the Roc curve (AUC)=0.926

The correlations between PTGVLFi and cardiac autonomic neuropathy (CAN) score were r=0.64 (P.0.0001).

The PTGi had a sensitivity of 92% and specificity of 80% (cut-off score >35.5) with the area under the curve=0.92) to detect diabetes.

The PTGVLFi had a sensitivity of 92% and specificity of 87% (cut-off score >25.5) with the area under the curve=0.91 to detect diabetes.

The correlations between the OGTT and PTGi were: r=−0.56 (p=0.003) for glucose, r=−0.41 (p=0.04) for insulin, and r=−0.50 (p=0.01) for insulin C-peptide.

The invention is described herein with reference to the accompanying figures wherein

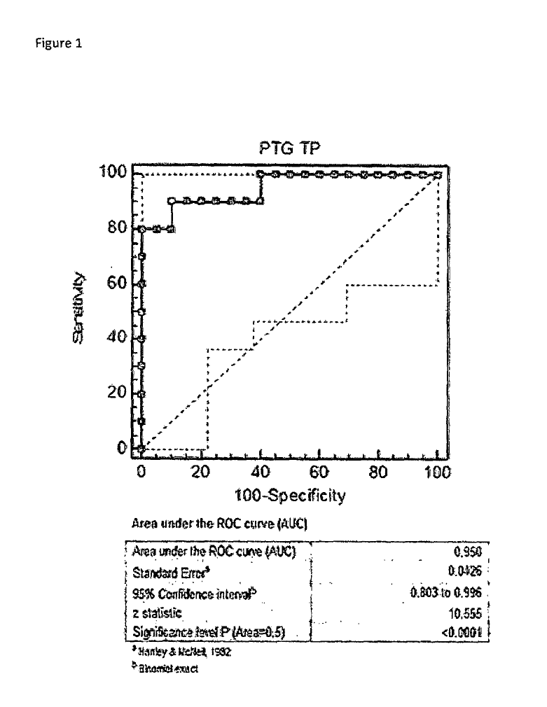

FIG. 1 illustrates the specificity and specificity of PTG TP to detect insulin resistance M value <4.5 (P.0.0001). M value <4.5 (P.0.0001).

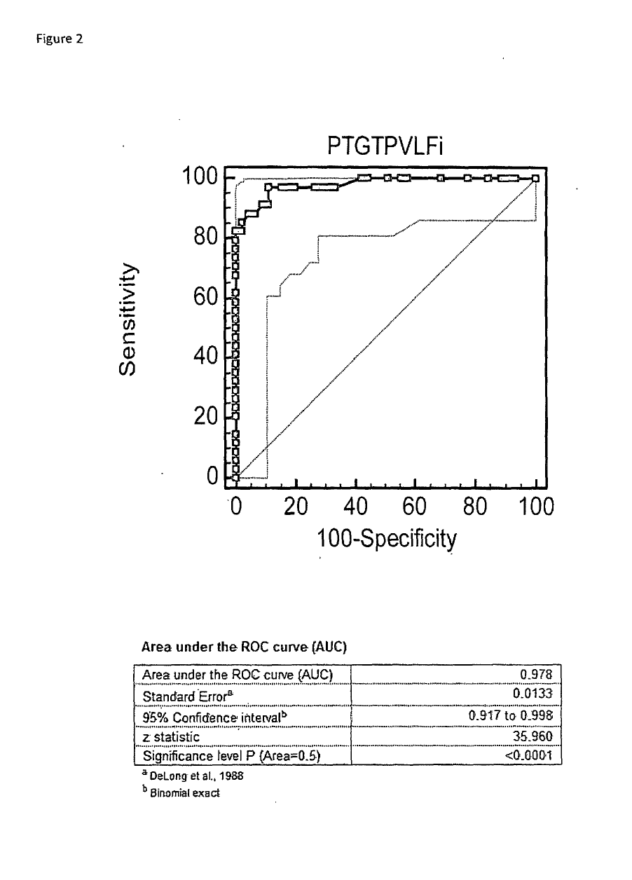

FIG. 2 illustrates the specificity and sensitivity of PTGVLFi to detect diabetes (P.0.001).

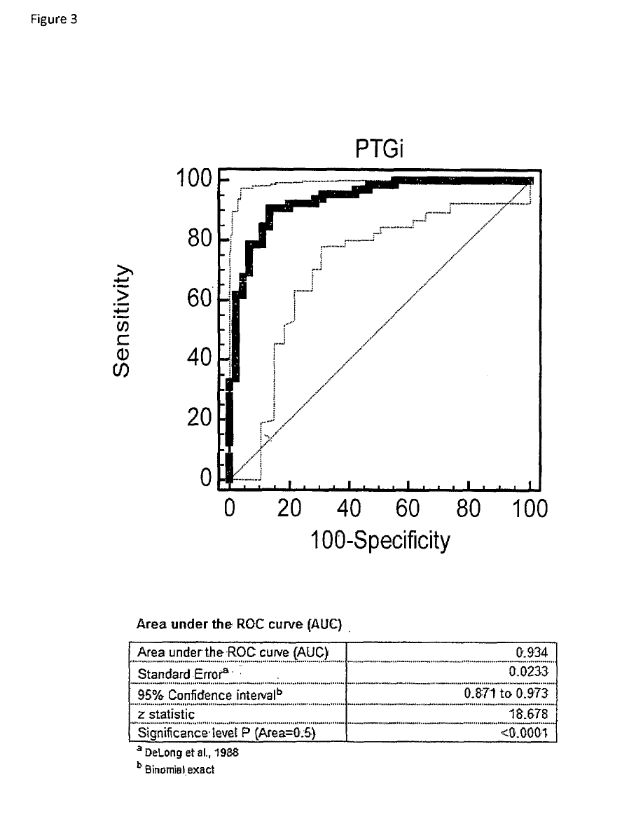

FIG. 3 illustrates the specificity and sensitivity of PTGi to detect atherosclerosis (P.0.001).

Studies

General Apparatus.

Each of the studies described used the TM-Oxi system which was used to measure a new parameter calculated with the Fast Fourier Transforms of the oximeter wave form (plethysmograph). The TM-Oxi system uses an oximeter and blood pressure device powered by the USB port of a PC.

The oximeter placed on the index finger of an individual has the ability to display in real time the photoelectrical plethysmography that represents the arterial blood volume changes during the cardiac cycle. Signal processing analysis of the waveform allows determination of the heart rate, the heart rate variability analysis and stiffness or aging index that is inversely proportional to the arterial compliance.

The spectral analysis using the Fast Fourier Transformation (FFT) of the first derivative of total records of the plethysmograph provides 3 frequencies; high, low and very low frequencies, the sum of the 3 frequencies is the Total Power of the spectral analysis and this is named Plethysmograph Total Power (PTG TP).

Although the studies use the TM-Oxi system the oximeter readings from other devices could be extrapolated and analyzed as described herein and therefore the present invention is not limited to the use of the TM-Oxi device but relates to the novel and inventive method of analysis of the plethysmograph described.

Study 1 examined Insulin resistance detection using spectral analysis of arterial plethysmography versus Euglycemic Hyperinsulinemic Clamp. This was carried out by Aglecio L. Souza and others at UNICAMP University Campinas Brazil.

Method: Thirty patients (23 women) in general good health of mean age 32 (range 22-55) years and BMI of 27.3 (range 19-49) Kg·m2, who were candidates for insulin resistance test were included in the study, and underwent hyperinsulinemic euglycemic clamp (HE clamp) test and examination with the TM-Oxi system. The TM-Oxi system uses an automatic blood pressure device and an oximeter managed by software, but in this study with focus on signal processing analysis of the oximeter data in spectral analysis. We investigated the cross-sectional association between insulin resistance (M value, assessed using (HE clamp) and the spectral analysis of the total records of the photoelectrical plethysmograph (PTG).

Statistical analysis was performed to correlate M value and PTG Total Power (PTG TP) using Brand Altman Plot. Receiver-operating characteristic curves were also constructed to determine the specificity and sensitivity of PTG TP, Body Mass Index (BMI) and blood glucose in detecting M value <4.5.

Results: The Spearman's coefficient of rank correlation (rho) was −0.624 (P. 0.001). PTG TP had a sensitivity of 90% and specificity of 90% (cutoff #370 m/s²) Area under the Roc curve (AUC)=0.95 to detect M value <4.5 (P.O.OOO1). BMI had a sensitivity of 80% and specificity of 60% (cutoff #28.8 Kgm2) AUC=0.752 to detect M value <4.5 (P.O.O1). Blood glucose had a sensitivity of 60% and specificity of 95% (cutoff #89.4) AUC=0.810 to detect M value <4.5 (P.0.001).

Conclusion: PTG TP parameter has the best AUC (0.95) comparing with the other existing available tests to detect the M value <4.5 of the HE clamp. Therefore, PTG TP provided by the TM-Oxi system represents a novel parameter of screening and follow ups for insulin resistance on large scale population. This parameter is independent factor of risk for T2DM and cardiovascular diseases. Such a tool, which is easy to use, non-invasive, and cost-effective, would be of great benefit for the control of pandemic diabetes diseases and its complications. A new study is underway to confirm the results with 100 patients.

Study 2 looked at a new approach in treatment management and early detection of foot neuropathy in diabetic population and was carried out by Pratiksha G Gandhi and others in Mumbai, India

Background: The new ADA and ESDA guidelines show the complexity of diabetes treatment, and also the prevention of diabetes complications. The ACCORD study suggests that tight control using AIC <=6.5% actually increases the risk for cardiovascular mortality associated with hypoglycemia.

Therefore, the new recommended 1 AC level was increased to 7%, and the algorithm treatments based only on 1 AC are considered controversial. In this context, new markers in adjunct of AIC in diabetes treatment management and early detection of complications will be useful.

Materials and Method:

One hundred sixty four patients were included in the study.

The patients were separated in 6 groups:

Group 1: One hundred two patients (70 males), with the mean age of 56 years (range 26-90), BMI 29 who were diagnosed with diabetes and undergoing treatment (Group 1)

Group 2: It is a subgroup of the group 1 comprising twenty five patients (26 males) with the mean age of 66 years (range 56-88) who were diagnosed with diabetes undergoing a treatment and signs and symptoms of tingling, burning or electric like pain or Extreme sensitivity to touch in feet

Group 3: It is a subgroup of the group 1 comprising sixty eight patients (42 males) with the mean age of 45 years (range 25-85) who were diagnosed with diabetes undergoing a treatment and without signs or symptoms of feet neuropathy.

Group 4: It is a subgroup of the group 1 comprising thirty one patients (23 males) with the mean age of 65 years (range 47-90) who were diagnosed with diabetes undergoing a treatment and with signs or symptoms of autonomic neuropathy such as muscle weakness or fatigue or Heat or exercise intolerance or bowel, bladder or digestive problems or changes in blood pressure, causing dizziness or lightheadedness.

Group 5: It is a subgroup of the group 1 comprising 71 patients (49 males) with the mean age of 56 years (range 26-85) who were diagnosed with diabetes undergoing a treatment and without signs or symptoms of autonomic neuropathy.

Group 6: Sixty two patients with the mean age of 40 years (range 22-85) who are in good condition without diabetes detected or signs of symptoms of foot neuropathy or autonomic neuropathy.

All the group patients underwent physical examination, questionnaire about know diseases, current treatment, history and symptoms according to the Michigan Neuropathy assessment and exam with the TM-0 xi system and SudoPath system. The TM-Oxi system provides a scoring card for cardiometabolic risk factors (CMR Score), autonomic neuropathy risk (ANR Score), Endothelial dysfunction (EndoT Score) and also frequencies of spectral analysis oximeter waveform (Photoplethysmography or PTG frequencies).

The SudoPath system uses a galvanic skin response technology in assessing the sudomotor function with a specific measurement process. It allows detection of skin microcirculation disorders, sweat glands density, and Latency of the response. The system provides a sudomotor response Score (SMR Score) based on these 3 parameters for early detection of peripheral foot neuropathy.

We compared:

1. Groups 1 to group 6 using the PTG very low frequency index (PTG VLFi), CMR score and EndoTscore.

2. Groups 2 and 3 using SMR Score

3. Groups 2 and 6 using SMR Score

4. Groups 4 and 5 and using ANR Score

5. Groups 4 and 6 using ANR Score

Statistical analysis was performed using Receiver-operating characteristic (ROC) curves to determine: 1. The specificity and sensitivity PTGVLFi and CMR Score as markers of Diabetes and EndoT score as marker of macrocirculation complication in diabetics patients comparing diabetes patients group and healthy subjects.

2. The specificity and sensitivity of SMR Score in detecting early foot neuropathy signs and symptoms comparing diabetes patients groups, and as marker of microcirculation complication in diabetic patients comparing diabetes patients group and healthy subjects.

3. The specificity and sensitivity ANR Score in detecting autonomic neuropathy signs and symptoms comparing diabetes patients groups, and as marker of autonomic nervous system complication in diabetic patients comparing diabetes patients group and healthy subjects.

Results:

Comparing diabetes patients group and healthy subjects, PTGVLFi had a sensitivity of 96% and specificity of 93.6% (cutoff #>26) to detect diabetes (P.O.OOO1). Area under the Roc curve (AUC)=0.989

Comparing diabetes patients group and healthy subjects group, CMR Score had a sensitivity of 91.2% and specificity of 90% (cutoff #>4) to detect diabetes (P.0.0001). Area under the Roc curve (AUC)=0.962

Comparing diabetes patients group and healthy subjects group, EndoT Score had a sensitivity of 88.2% and specificity of 88.6% (cutoff #>1) to detect diabetes (P.0.0001). Area under the Roc curve (AUC)=0.962

Comparing diabetes patients group with symptoms of foot neuropathy and diabetes patients group without symptoms of foot neuropathy, SMR Score had a sensitivity of 91.4% and specificity of 79.1% (cutoff #>3) to detect foot neuropathy symptoms in diabetic patients (P.0.0001). Area under the Roc curve (AUC)=0.858

Comparing diabetes patients group with symptoms of foot neuropathy and healthy subjects group, SMR Score had a sensitivity of 91.4% and specificity of 96.8% (cutoff #>3) to detect foot neuropathy symptoms in healthy subject (P.0.0001). Area under the Roc curve (AUC)=0.982

Comparing diabetes patients group with symptoms of autonomic neuropathy and diabetes patients group without symptoms of autonomic neuropathy, ANR Score had a sensitivity of 69.4% and specificity of 86.3% (cutoff #>7) to detect autonomic neuropathy in diabetic patients (P.O.OOO1). Area under the Roc curve (AUC)=0.831

Comparing diabetes patients group with symptoms of foot neuropathy and healthy subjects group, ANR Score had a sensitivity of 87.2% and specificity of 95.1% (cutoff #>5) to detect autonomic neuropathy in healthy subjects (P.0.0001). Area under the Roc curve (AUC)=0.964

Conclusion: PTGVLFi and CMR Scores provided by the TM-Oxi system have very high sensitivity and specificity to detect diabetes and should be used as new markers in screening and treatment management of diabetic patients.

Comparing Diabetes patients and healthy subjects, SMR score, ANR Score and EndoTscore have a high sensitivity and specificity to detect diabetes complications such as respectively foot neuropathy symptoms, autonomic neuropathy symptoms and endothelial dysfunction.

Comparing the diabetes patients with and without foot pains or autonomic neuropathy symptoms, SMR score and ANR score will be useful in early detection of such complications in diabetes patients.

In conclusion, in one hand these results will be a useful tool to assess the susceptibility of patients with risk factors, and will also ensure better monitoring of diabetes treatment in adjunct of AIC percent, and in second hand to assess the susceptibility of patients with risk factors of diabetes complications, thus reducing their occurrence in the long term.

These findings have to be confirmed by large scale studies using TM-Oxi and SudoPath system.

Study 3 related to spectral analysis of photoplethysmography in screening of atherosclerosis and was carried out by Dr Pratiksha G Gandhi, Cardiologist in Mumbai, India.

Background: Atherosclerosis is a leading cause of cardiovascular death due to the increasing prevalence of the disease and the impact of risk factors such as diabetes, obesity or smoking Sudden cardiac death is the primary consequence of coronary artery disease in 50% of men and 64% of women. Currently the only available strategy to reduce mortality in the at-risk population is primary prevention; the target population must receive screening for atherosclerosis. The value of screening for subclinical atherosclerosis is still relevant, and it has become standard clinical practice with the emergence of noninvasive techniques (radio frequency, measurement of intima-media thickness, and flow-mediated vasodilatation). In this study we present a new non-invasive technique based upon the spectral analysis of the plethysmography provided by an oximeter.

Material and Method:

Sixty-three patients (12 women), with the mean age of 62.9 years (range 40-80) who were diagnosed with atherosclerosis using CAG report (Group 1) and forty-seven subjects (13women) with the mean age of 45.1 years (range 25-85) who are supposed healthy (group 2), were included in the study.

The group 1 was separated into 2 subgroups

Subgroup 1 A: Atherosclerosis patients without surgery such as coronary artery bypass grafting (CABG) or Coronary angioplasty also called percutaneous coronary intervention (PCI),

Subgroup IB: Atherosclerosis patients with surgery (CABG or PCI).

These patients and subjects underwent examination with the TM-Oxi system. The TM-Oxi system uses a blood pressure device and oximeter, and the focus of this study was on the signal processing analysis of the oximeter waveform (Photoplethysmography or PTG) and a scorecard based on this analysis (EndoT Score). We compared the 2 groups 1 and 2 using the PTG spectral analysis Index (PTGi) and the EndoT Score.

We compared also the 2 subgroups 1 A and IB using the PTG very low frequency (PTG VLF).

Statistical analysis was performed using Receiver-operating characteristic curves (ROC) to determine:

The specificity and sensitivity of PTGi and EndoTscore in detecting atherosclerosis comparing group 1 and group 2.

The specificity and sensitivity of PTGVLF in detecting atherosclerosis patients undergoing in Surgery comparing the subgroup 1 A and IB

Results: PTGi had a sensitivity of 86.1% and specificity of 87.3% (cutoff #<40.8) to detect atherosclerosis (P.0.0001). Area under the Roc curve (AUC)=0.926

EndoTscore had similar results with sensitivity of 86.2% and specificity of 88.2% (cutoff >1) to detect atherosclerosis (P.0.0001). Area under the Roc curve (AUC)=0.902

PTGVLF had a sensitivity of 82.6% and specificity of 100% (cutoff #<69) to detect atherosclerosis patient undergoing in Surgery such as CABG or PCI (P.0.0001). Area under the Roc curve (AUC)=0.952

Conclusion: PTGi parameter and EndoT Score have high sensitivity and specificity to detect atherosclerosis and will be useful as new markers of the endothelial dysfunction.

PTG VLF has a good sensitivity and remarkable 100% of specificity to detect the benefits of coronary surgery. TM-Oxi parameter and Score will be a useful tool to assess the susceptibility of patients with risk factors, and ensures better monitoring of atherosclerosis and surgery, thus reducing the occurrence of cardiovascular events in the long term.

Study 4 Lewis et al. A cross-sectional assessment to detect Type 2 Diabetes with endothelial and autonomic nervous system markers using a novel system. Journal of Diabetes & metabolic disorders, 2014, 13:118

Background: Type 2 diabetes mellitus is frequently unrecognized until complications appear. Diabetic autonomic neuropathy is one of the early complications of type 2 diabetes mellitus, resulting in autonomic nervous system (ANS) dysfunction. The purpose of this study was to determine the validity of ANS function indicators to screen for type 2 diabetes mellitus, as measured by the TM-Oxi and SudoPath system.

Methods: All enrolled participants completed a basic sociodemographic and medical history questionnaire including current medications. Healthy controls (n=25) underwent a 2-hour oral glucose tolerance test (OGTT) to evaluate glucose, insulin, and insulin C-peptide. Patients with type 2 diabetes mellitus (n=24) were assessed with fasting plasma glucose (FPG) and glycosylated hemoglobin. The TM-Oxi and SudoPath system evaluation was completed by all subjects. Data were analyzed using SPSS 22. Frequency and descriptive statistics were calculated on all variables. The criterion for statistical significance was α=0.05.

Results: The twenty-five healthy controls had a mean age of 37.0 years. The twenty-four type 2 diabetes mellitus patients currently undergoing standard treatment had a mean age of 48.9 years. Based on the American Diabetes Association guidelines, we detected pre-diabetes in 4 subjects and diabetes in 1 subject, while all other subjects had normal FPG values. At 120 minutes, the correlations between the OGTT and cardiometabolic risk score (CMRS) were:

r=0.56 (p=0.004) for glucose and r=0.53 (p=0.006) for insulin. At 120 minutes, the correlations between the OGTT and photoplethysmography index (PTGi) were: r=−0.56 (p=0.003) for glucose and r=−0.41 (p=0.04) for insulin. The CMRS, PTGi, and plethysmography total power index (PTGVLFi) differed significantly between the diabetes patients and healthy participants. The specificity and sensitivity for the CMRS, PTGi, and PTVLFi comparing the diabetes patients with healthy controls were high.

Conclusion: The TM-Oxi and SudoPath system shows promise as a valid, convenient, and non-invasive screening method for type 2 diabetes mellitus. The ANS function and CMR indicators measured by this system may be useful in guiding diabetes and cardiovascular health screening, treatment, and monitoring.

Study 5. Gandhi PG, Rao GH. The spectral analysis of the photoplethysmography to evaluate an independent cardiovascular risk. Int J Gen Med. 2014 Dec. 9; 7:539-47.

Background

In this study, we evaluate homeostatic markers correlated to autonomic nervous and endothelial functions in a population of coronary artery disease (CAD) patients versus a control group. Since CAD is the highest risk marker for sudden cardiac death, the study objective is to determine whether an independent cardiovascular risk score based on these markers can be used alongside known conventional cardiovascular risk markers to strengthen the understanding of a patient's vascular state.

Materials and Methods:

Sixty-five subjects (13 women) with a mean age of 62.9 years (range 40-80 years) who were diagnosed with CAD using coronary angiography (group 1) and seventy-two subjects (29 women) with a mean age of 45.1 years (range 18-85 years) who claimed they were healthy (group 2) were included in the study. These subjects underwent examination with the TM-Oxi and SudoPath systems at IPC Heart Care Centers in Mumbai, India. The TM-Oxi system takes measurements from a blood pressure device and a pulse oximeter. The SudoPath measures galvanic skin response to assess the sudomotor pathway function. Spectral analysis of the photoplethysmograph (PTG) waveform and electrochemical galvanic skin response allow the TM-Oxi and SudoPath systems to calculate several homeostatic markers, such as the PTG index (PTGi), PTG very low frequency index (PTGVLFi), and PTG ratio (PTGr). The focus of this study was to evaluate these markers (PTGi, PTGVLFi, and PTGr) in CAD patients against a control group, and to calculate an independent cardiovascular risk factor score: the PTG cardiovascular disease risk score (PTG CVD), calculated solely from these markers. We compared PTGi, PTGVLFi, PTGr, and PTG CVD scores between the CAD patient group and the healthy control group. Statistical analyses were performed using receiver operating characteristic curves to determine the specificity and sensitivity of the markers to detect CAD at optimal cutoff values for PTGi, PTGVLFi, PTGr, and PTG CVD. In addition, correlation analyses between these markers and conventional autonomic nervous system and endothelial function markers were performed to understand the possible underlying physiological sources of the differences observed in marker values between CAD patients and healthy control patients. Additionally, t-tests were performed between two subgroups of the CAD patient group to determine whether diabetic or coronary artery bypass grafting (CABG) patients have significantly different PTGi marker values.

Results:

Each spectral analysis PTG marker yielded a high specificity and sensitivity to detect CAD. Most notably, the PTG CVD score had a sensitivity of 82.5% and specificity of 96.8%, at a cutoff of 2, when used to detect CAD (P=0.0001; area under the receiver operating characteristic curve =0.967). The PTG spectral analysis markers were well-correlated to other autonomic nervous system and endothelial function markers. CAD diabetic patients (n=27) had a lower PTGi value compared with the CAD non-diabetic patients (n=38): and patients that underwent CABG (n=18) had a higher PTGi value compared with the CAD without CABG surgery patients (n=47).

Conclusion

The spectral analysis of the photoplethysmography method is noninvasive, fast, operator-independent, and cost-effective, as only an oximeter and galvanic skin response device are required in order to assess in a single testing the autonomic nervous system and endothelial function. The spectral analysis techniques used on the photoplethysmogram, as outlined in this study, could be useful when used alongside conventional known cardiovascular disease risk markers.

### REFERENCES

King H, Aubert R E, Herman W H, Global burden of diabetes, 1995-2025: prevalence, numerical estimates, and projections. Diabetes Care 21 : 1414 1998.
American Diabetes Association. Economic costs of diabetes in the US in 2007. Diabetes Care 31 : 1-20, 2008
Reaven, G. M. Pathophysiology of insulin resistance in human disease. Physiol. Rev. 75:473-486, 1995
Abdul-Ghani M A, Williams K, DeFronzo R A, Stern M: What is the best predictor of future type 2 diabetes? Diabetes Care 30: 1544-1548, 2007
Bonora E, Formentini G, Calcaterra F. HOMA-estimated insulin resistance is an independent predictor of cardiovascular disease in type 2 diabetic subjects. Diabetes Care; 25: 1 135-1 141, 2002.
DeFronzo R A Insulin resistance: a multifaceted syndrome responsible for NIDDM, obesity, hypertension, dyslipidaemia and atherosclerosis. Neth J Med. 50: 191-7, 1997
Abbasi F, Brown BWB, Lamendola C, McLaughlin T, Reaven G M. 2002. Relationship between obesity, insulin resistance, and coronary heart disease risk. J. Am. Coll. Card. 40:937-43, 2002
Nathan D M, Cleary P A, et al. Diabetes Control and Complications Trial/Epidemiology of Diabetes Interventions and Complications (DCCT/EDIC) Study Research Group. Intensive diabetes treatment and cardiovascular disease in patients with type 1 diabetes. N Engl J Med. 353 :2643-53,2005
Barbara B. Kahn and Jeffrey S. Flier. Obesity and insulin resistance. The Journal of Clinical Investigation; 106(4):473-481,2000
Stratton I M, Adler A I, Neil H A W, Matthews D R, Manley S E, Cull C A, Hadden D, Turner R C, Holman R: Association of glycaemia with macrovascular and microvascular complications of type 2 diabetes (UKPDS 35): prospective observational study: BMJ 321 :405-412, 2000
Freeman R: The peripheral nervous system and diabetes. In Joslin's Diabetes Mellitus. Weir G, Kahn R, King G L, Eds. Philadelphia, Lippincott, 2002
UK Prospective Diabetes Study Group. Overview of 6 years' therapy of type II diabetes: a progressive disease (UKPDS 16). Diabetes. 1995; 44: 1249-1258.
Marshall S M, Prevention and early detection of vascular complications of diabetes. BMJ 2006; 333
The Diabetes Control and Complications Trial Research Group. The effect of intensive treatment of diabetes on the development and progression of long-term complications in insulin-dependent diabetes mellitus. N Engl J Med. 1993; 329(14):977-986.
DeFronzo R A, Tobin J D, Andres R. Glucose clamp technique: a method for quantifying insulin secretion and resistance. Am J Physiol. 1 September; 237(3):E214-23.1979
Lindsey George, Fida Bacha, SoJung Lee, Hala Tfayli, Elisa Andreatta, and Silva Arslanian. Surrogate Estimates of Insulin Sensitivity in Obese Youth along the Spectrum of Glucose Tolerance from Normal to Prediabetes to Diabetes. Clin Endocrinol Metab, 96(7):2136-2145,201 1
Millasseaua S C, Rittera J M, Takazawab K, et al. Contour analysis of the photoplethysmographic pulse measured at the finger. J Hypertens. 2006;24:1449-56.
Brillante D G, O'Sullivan A J, Johnstone M T, et al. Arterial stiffness and haemodynamic response to vasoactive medication in subjects with insulin-resistance syndrome. Clin Sci. 2008; 1 14: 139-47.
American Diabetes Association. Diabetes Care, 2004, 27(1); 51 1-514
UK Prospective Diabetes Study Group. Overview of 6 years' therapy of type II diabetes: a progressive disease (UKPDS 16). Diabetes. 1995;44: 1249-1258.
UK Prospective Diabetes Study Group. Intensive blood-glucose control with sulphonylureas or insulin compared with conventional treatment and risk of complications in patients with type 2 diabetes (UKPDS 33).Lancet. 1998;352:837-853.
Tuomilehto J, Lindstrom J, Eriksson J G, Valle T T, Hamalainen H, Ilanne-Parikka P, Keinanen-Kiukaanniemi S, Laakso M, Louheranta A, Rastas M, Salminen V, Uusitupa M: Prevention of type 2 diabetes mellitus by changes in lifestyle among subjects with impaired glucose tolerance. N Engl J Med 344: 1343-1350, 2001
Knowler W C, Barrett-Connor E, Fowler S E, Hamman R F, Lachin J M, Walker E A, Nathan D M: Reduction in the incidence of type 2 diabetes with lifestyle intervention or metformin. N Engl J Med 346:393-403, 2002
Gerstein H C, Yusuf S, Bosch J, Pogue J, Sheridan P, Dinccag N, Hanefeld M, Hoogwerf B, Laakso M, Mohan V, Shaw J, Zinman B, Holman R R: Effect of rosiglitazone on the frequency of diabetes in patients with impaired glucose tolerance or impaired fasting glucose: a randomised controlled trial. Lancet 368: 1096-1 105, 2006
DeFronzo R A, Tripathy D, Schwenke D C, Banerji M, Bray G A, Buchanan T A, Clement S C, Henry R R, Hodis H N, Kitabchi A E, Mack W J, Mudaliar S, Ratner R E, Williams K, Stentz F B, Musi N, Reaven P D; ACT NOW Study. Pioghtazone for diabetes prevention in impaired glucose tolerance. N Engl J Med. 364: 1 104-15, 2011.
Fleischman A and Rhodes E T. Management of obesity, insulin resistance and type 2 diabetes in children: consensus and controversy. Diabetes, Metabolic Syndrome and Obesity: Targets and Therapy 2009:2 185-202
Reaven G M. The insulin resistance syndrome: definition and dietary approaches to treatment. Annu Rev Nutr. 25:391-406, 2005
American Diabetes Association. Management of Hyperglycemia in Type 2 Diabetes: A Patient-Centered Approach. Diabetes Care 2012;35: 1364-1379 European Association for the Study of Diabetes. Management of Hyperglycemia in Type 2.
Diabetes Diabetologia 2012; 55: 1577-1596.
Study 4 Lewis et al. A cross-sectional assessment to detect Type 2 Diabetes with endothelial and autonomic nervous system markers using a novel system. Journal of Diabetes & metabolic disorders, 2014, 13:118
Gandhi P G, Rao G H. The spectral analysis of the photoplethysmography to evaluate an independent cardiovascular risk. Int J Gen Med. 2014 Dec. 9; 7:539-47

1. A method to detect insulin resistance, diabetes and/or cardiovascular or autonomic neuropathy complications, the method comprising performing a spectral analysis using Fast Fourier Transformation (FFT) of the first derivative of total records of an oximeter wave form (plethysmograph), to obtain 3 frequencies; high (PTGHF), low (PTGLF) and very low (PTGVLF) frequencies wherein the sum of the 3 frequencies is the total power of the spectral analysis in ms² (PTG TP) and wherein the sum of the amplitude of the 3 frequencies is the PTG Index of the Spectral Analysis (PTGi).

2. The method of claim 1 wherein PTG TP >370 m/s² is a marker for insulin resistance.

3. The method of claim 1 wherein PTGi <40 is a marker of diabetes and endothelial dysfunction or atherosclerosis.

4. The method of claim 1 wherein PTGVLFi is calculated with an algorithm using PTG VLF and a marker of the sudomotor function using a galvanic skin response device wherein PTGVLFi >33 is a marker for diabetes and cardiac autonomic neuropathy.

5. The method of claim 1 wherein the measurements are carried out using an oximeter and a galvanic skin response device.

6. The method of claim 2 wherein the measurements are carried out using an oximeter and a galvanic skin response device.

7. The method of claim 3 wherein the measurements are carried out using an oximeter and a galvanic skin response device.

8. The method of claim 4 wherein the measurements are carried out using an oximeter and a galvanic skin response device.

9. Use of an oximeter and galvanic skin response device in the method of claim 1.

10. Use of an oximeter and galvanic skin response device in the method of claim 2.

11. Use of an oximeter and galvanic skin response device in the method of claim 3.

12. Use of an oximeter and galvanic skin response device in the method of claim 4.

13. Use of an oximeter and a galvanic skin response device in the detection of insulin resistance, diabetes and cardiovascular complications such as atherosclerosis and autonomic neuropathy.

14. An apparatus for use in the detection of insulin resistance, diabetes and/or cardiovascular or autonomic neuropathy complications, the apparatus comprising software installed on a PC and an oximeter wherein the software is capable of performing a fast Fourier transformation of the first derivative of total records of a plethysmograph trace from the oximeter and provides the frequencies of the resulting spectral analysis of high (PTGHF), low (PTGLF) and very low frequencies (PTGVLF) and the total power PTGTP (the sum of the surface of the 3 frequencies in ms²⁾ (meters per second squared) and the sum of the amplitude of the 3 frequencies of the spectral analysis, the PTGi.

15. The apparatus of claim 14 wherein the software calculates the PTGVLF Index using a combination of oximeter and a galvanic skin response device.

16. The apparatus of claim 15 wherein the apparatus is the TM-Oxi system and SudoPath hardware and software.

17. The apparatus of claim 14 wherein the apparatus is the TM-Oxi system and SudoPath hardware and software.
